(19) United States
(12) Patent Application Publication
(10) Pub. No.: US 2007/0135699 A1
(43) Pub. Date: Jun. 14, 2007

(54) BIOSENSOR WITH ANTIMICROBIAL AGENT

(75) Inventors: W. Kenneth Ward, Portland, OR (US); Richard Sass, Portland, OR (US); Mark Neinast, Portland, OR (US); Robert Bruce, Portland, OR (US)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: iSENSE CORPORATION, Portland, OR (US)

(21) Appl. No.: 11/609,768

(22) Filed: Dec. 12, 2006

Related U.S. Application Data
(60) Provisional application No. 60/749,715, filed on Dec. 12, 2005.

(51) Int. Cl. A61B 5/04; A61B 5/05

(52) U.S. Cl. 600/373; 600/396; 600/345

(57) ABSTRACT

Embodiments of the present invention provide methods, apparatuses, and systems for sensing analyte with devices having antimicrobial properties. Embodiments of the present invention provide specialized coatings or specialized materials that reduce the risk of tissue infection around biosensors that reside fully or partially within subcutaneous tissue or within a blood vessel.

### CROSS REFERENCE TO RELATED APPLICATIONS

The present application claims priority to U.S. Provisional Patent Application No. 60/749,715, filed Dec. 12, 2005, entitled “Biosensor with Antimicrobial Agent,” the entire disclosure of which is hereby incorporated by reference in its entirety.

### TECHNICAL FIELD

Embodiments of the present invention relate to the field of medical devices, and, more specifically, to biosensors having antimicrobial properties.

### BACKGROUND

The prognosis for persons with diabetes is related to the degree of diabetes control. The best prognosis is for those who are able to keep their blood glucose levels within, or close to, the normal range of 75-115 mg/dl. In contrast, persons who allow their glucose levels to stay at elevated levels for a prolonged period of time risk such chronic complications as eye disease (retinopathy), kidney disease (nephropathy), cardiovascular disease and disease of the nerves and feet. Typically, in order to monitor their glucose levels, individuals with diabetes use a sharp lancet to obtain blood from their fingertip, then place the blood on a strip that is read by a portable meter. This process, one type of discrete glucose monitoring, is painful and expensive and must be repeated a number of times each day for the patient to be well-informed about his or her level of diabetes control.

More recently, devices have been developed that allow persons with diabetes to monitor their glucose levels much more frequently and with less discomfort. Such devices include continuous glucose monitors or continuous glucose sensors. Some of these devices are elongated glucose sensing structures that may be placed through the skin into the subcutaneous tissue. In such a configuration, the area of the sensor that measures glucose may be situated in the subcutaneous tissue and may respond to glucose that is present in interstitial fluid.

The signal that is derived from the glucose concentration (which may be an electrical current, electrical potential, electrical charge, optical signal, or other signal) may be transferred through a conductor in or on the sensor device and into circuitry.

When the signal reaches, for example, a portion of the unit above the surface of the skin, it may be stored in memory or it may be transmitted to a remote receiver, where the user may interpret the signal and may utilize the information in order to improve his or her degree of diabetes control.

One problem with sensors that pierce the skin is that the resulting puncture wound may become colonized or infected. The process of colonization refers to a growth of microorganisms such as bacteria, fungi, or viruses that occurs in the absence of clinical symptoms. The term infection means that such growth has continued beyond simple colonization to the point where clinical symptoms (apparent to the patient) or clinical signs (apparent to the health care professional) have occurred. An infection is more serious than a colonization, but ideally, both should be prevented. As compared to other types of wounds, puncture wounds are notable for often leading to colonization or infection.

One reason that such wounds may become infected, for example in humans, is that normal humans have bacteria and other microorganisms on their skin. During a puncture wound, such as that which occurs during introduction of a biosensor, these bacteria may become introduced (inoculated) into the deeper subcutaneous tissues. This is a particular problem for patients whose skin is normally colonized with spherical bacteria such as staphylococci or streptococci. These types of bacteria are common causes of infection of the skin and subcutaneous structures. Infections of the skin include boils, pustules, cellulitis and erysipelas. Erysipelas is an infection of the most superficial portion of the skin whereas cellulitis includes deeper structures including subcutaneous tissues. Symptoms of skin infection often include redness, fever, local heat, pus and pain. Infections may occur even when the skin has been sterilized prior to sensor insertion.

### BRIEF DESCRIPTION OF THE DRAWINGS

Embodiments of the present invention will be readily understood by the following detailed description in conjunction with the accompanying drawings. Embodiments of the invention are illustrated by way of example and not by way of limitation in the figures of the accompanying drawings.

FIG. 1 illustrates an exemplary biosensor device in accordance with an embodiment of the present invention;

FIG. 2 illustrates an on-skin unit for a biosensor in accordance with an embodiment of the present invention; and

FIG. 3 illustrates an exemplary attachment mechanism to attach an on-skin unit to a patch in accordance with an embodiment of the present invention.

### DETAILED DESCRIPTION OF EMBODIMENTS OF THE INVENTION

In the following detailed description, reference is made to the accompanying drawings which form a part hereof, and in which is shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present invention.

For the purposes of the description, a phrase in the form “A/B” means A or B. For the purposes of the description, a phrase in the form “A and/or B” means “(A), (B), or (A and B)”. For the purposes of the description, a phrase in the form “at least one of A, B, and C” means “(A), (B), (C), (A and B), (A and C), (B and C), or (A, B and C)”. For the purposes of the description, a phrase in the form “(A)B” means “(B) or (AB)” that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

In various embodiments of the present invention, methods, apparatuses, and systems for sensing analyte with devices having antimicrobial properties are provided. In exemplary embodiments of the present invention, a computing system may be endowed with processing components, memory, storage media, etc. and/or one or more components of the disclosed articles of manufacture and/or systems and may be employed to perform one or more sensing/analysis methods as disclosed herein.

Biosensors according to embodiments of the present invention may be elongated such as a wire type electrode/sensor or a catheter, but may be designed to measure a biological parameter rather than to simply deliver fluid or extract fluid from the body. In embodiments, suitable biosensors may be able to both measure analyte and to deliver fluid, such as a drug, to the body. In such an embodiment, a hollow sensor having one or more drug delivery lumens may be provided, and sensing may occur on an exterior surface or tissue-contact surface of the sensor.

Embodiments of the present invention provide specialized coatings or specialized materials that reduce the risk of tissue infection around biosensors that reside fully or partially within subcutaneous tissue or within a blood vessel. Embodiments of the present invention may be used with sensors that pierce the skin and reside in subcutaneous tissue (transcutaneous subcutaneous devices) and those that pierce the skin and reside in the vascular space (transcutaneous intravascular devices), whether fully or partially implanted, and regardless of the length of time the biosensor resides, or is intended to reside, in the subcutaneous tissue or vascular space.

In embodiments, various antimicrobial agents may be used on at least a portion of a biosensor to prevent or reduce infection. In an embodiment of the present invention, silver may be combined with or disposed on sensor materials in order to prevent tissue infection. In embodiments, silver may be in the form of silver ion, metallic silver, colloidal silver, silver salt, silver sulfadiazine or another form of silver. Silver is known to have antimicrobial characteristics.

In an embodiment of the present invention, another useful characteristic of silver or a silver layer is that silver chloride may be generated on the silver by a chemical technique such as immersion in ferric chloride or by passing a current through a solution that contains hydrochloric acid and/or potassium chloride. Silver chloride, when layered over silver, may serve as a combined reference electrode and counter electrode in a two-electrode amperometric system and may serve as a reference electrode in a three-electrode system.

In an embodiment, silver/silver chloride may exist on the outer part of a sensor that is in chemical communication with tissue and may serve both as a reference electrode (or combined reference electrode and counter electrode) and as an antimicrobial agent to minimize the risk for infection.

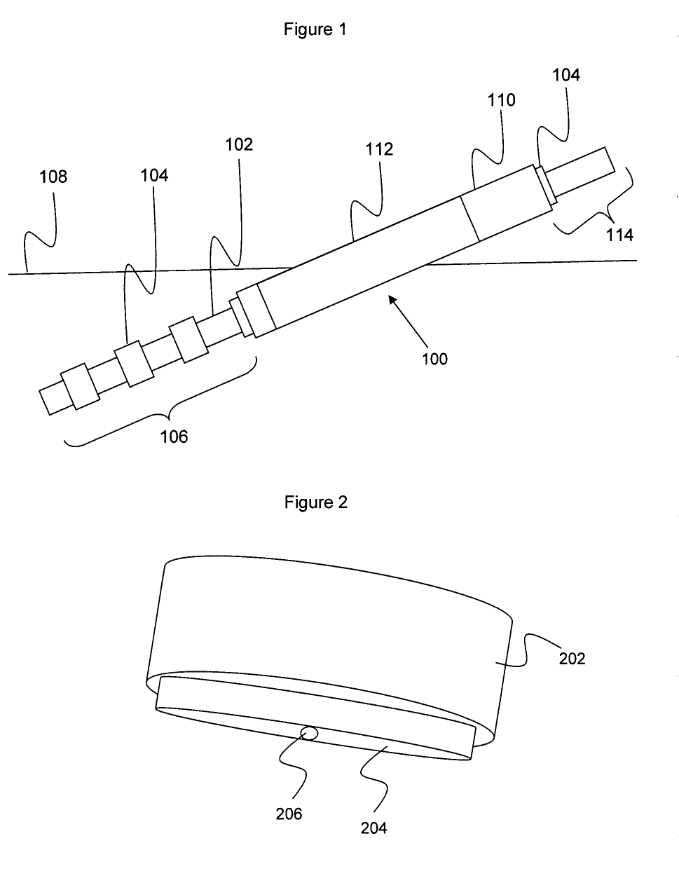

In an embodiment, an antimicrobial agent, such as silver, gold, etc., may be placed on a portion of a biosensor, for example, the portion that extends through the skin (the epidermis and possibly the dermis), as shown in FIG. 1. In an embodiment, an antimicrobial agent or a portion of a reference electrode having antimicrobial properties may extend along a biosensor from a location above the surface of the skin to a location below the surface of the skin, for example extending through the epidermis and into or through the dermis. In a separate embodiment, an antimicrobial agent may be placed over a longer segment of the sensor, for example, the entire part of the sensor that contacts tissue, preferably in such a way that there is little to no interference with the analyte sensing operations of the sensor.

In an embodiment of the present invention, at least part of a biosensor may be made from an antimicrobial agent. In embodiments, an antimicrobial metal such as silver may be applied to a portion of a biosensor in the form of a wrapped wire, whether the wire is cylindrical, flat, or having a different geometry, or may be applied as a thin film, or using another deposition process. In an embodiment in which silver (such as silver wire) is chloridized, the silver may be chloridized before or after application to the biosensor. In a further embodiment, the Ag/AgCl may be further treated, before or after application to the biosensor, to stabilize the chloridization layer.

FIG. 1 illustrates an exemplary biosensor device 100 in accordance with an embodiment of the present invention. Device 100 has a central, elongated sensor 102, which may be flexible or rigid. In an embodiment, a flexible sensor is one that may be flexed repeatedly, such as the type of flexion experienced by a subcutaneously implanted sensor in a human during normal movement, over a period of time (such as 3-7 days or more) without fracture. In an embodiment, a flexible sensor may be flexed hundreds or thousands of times without fracture. Sensor 102 may be constructed from a single material, or may be a combination of materials, such as a central core surrounded by at least one additional layer, whether the device is hollow or solid in construction. In an embodiment of the present invention, various types of biosensors may be utilized whether hollow or not, regardless of the materials (metals, polymers, fibers, etc.) used to construct the biosensor. Additional details of such sensors may be found in U.S. Pat. No. 7,146,202, the entire contents of which are hereby incorporated by reference. Sensor 102 may be surrounded in one or more locations with an insulating layer 104, for example shaped as a sleeve or as an insulating nub, such as constructed from polyimide or other insulating material.

In an embodiment, in region 106 of sensor 102 in the subcutaneously or intravenously implantable portion beneath skin surface 108, signal transduction membranes (not shown) may also be present.

In an embodiment, a biosensor may include a membrane or membrane system on at least a portion of the biosensor through which analyte, such as glucose or lactate, may be transported and/or measured. In an embodiment, such membranes may include an interferent reducing inner layer, an enzyme layer (such as for reacting with glucose or lactate), and a permselective outer layer. In an embodiment, a silane layer may be provided under and/or over the enzyme layer. Additional details of such membranes may be found in U.S. Pat. No. 5,165,407, US Patent Application Publication No. 2005/0004324, and U.S. patent application Ser. No. 11/404,528, the entire contents of which are hereby incorporated by reference.

In an embodiment, sensor 102 may serve as an indicating electrode. A reference electrode 110 may also be provided. Reference electrode 110 may also serve as a counter electrode in various embodiments. Reference electrode 110 may be constructed from a variety of materials, including silver or a silver/silver chloride (Ag/AgCl) bilayer. In an embodiment, an antimicrobial agent or sleeve 112 may be provided as a separate layer (separate material) or the same material as reference electrode 110 if reference electrode 110 is selected for having antimicrobial properties. Thus, in an embodiment, a reference electrode may be provided (with or without antimicrobial properties) with a separate overlaid antimicrobial agent (for example, in the form of a layer or sleeve). In another embodiment, a reference electrode may be constructed from a material having antimicrobial properties without the addition of a separate antimicrobial agent. Thus, in an embodiment, if a reference electrode of, for example, Ag/AgCl is utilized, an additional antimicrobial agent may be used, if desired, although such an additional layer is not necessary to impart antimicrobial properties to the device since the reference electrode material has such properties.

In an embodiment, sensor 102 and reference electrode 110 may be electrically coupled from terminal region 114 to an electrical network (not shown). The term “electrical network” means electronic circuitry and components in any desired structural relationship adapted to, in part, receive an electrical signal from an associated sensor and, optionally, to transmit a further signal, for example to an external electronic monitoring unit that is responsive to the sensor signal. The circuitry and other components may or may not include a printed circuit board, a tethered or wired system, etc. Signal transmission may occur over the air with electromagnetic waves, such as RF communication, or data may be read using inductive coupling. In other embodiments, transmission may be over a wire or via another direct connection.

In embodiments, the various layers presented in FIG. 1 may circumferentially surround the underlying layers as shown or may only partially cover or surround the underlying layers.

In an embodiment, silver or another antimicrobial agent may be deposited on a device by electroplating. In another embodiment, an antimicrobial agent may be deposited by an electroless process. In electroless deposition, the process is a chemical process instead of a process that uses an electric current to deposit metal. The electroless metal coating process deposits a uniform coating regardless of substrate shape, overcoming a major drawback of electroplating. In electroless coating, deposition thickness may be controlled simply by controlling immersion time. In another embodiment, an antimicrobial agent may be deposited by thermal evaporation of metal, which deposits a layer of the metal in a line-of-sight fashion.

In another embodiment, an antimicrobial agent may be deposited by a sputtering process in a plasma environment. In another embodiment, ion plating (ion implantation) may be used and, in such a method, the evaporant may be generated by thermal evaporation. In ion plating of, for example, silver, the silver ions may be implanted into the substrate, forming a tight bond.

In another embodiment, an antimicrobial agent may be coated by arc spraying, a thermal form of metal spraying using a wire arc gun. An electric arc liquefies the metal, and an air spray propels it onto the substrate.

In another embodiment, an antimicrobial agent may be applied by plasma spray metal coating, a process that relies on a hot, high-speed plasma flame (nitrogen, hydrogen, or argon) to melt a powdered material and spray it onto the substrate. A direct-current arc is maintained to excite gases into the plasma state.

In an embodiment, an antimicrobial agent such as silver may be deposited to form a monolayer or multilayer thin film. Suitable processes for depositing such a thin film include, sputtering, chemical vapor deposition, molecular beam epitaxy, sol gel processing, spin coating, pulsed laser deposition, etc.

Other mechanisms may be utilized to apply an antimicrobial agent to a device, such as a biosensor, for example dip coating, as desired for the particular application.

As indicated above, in an embodiment of the present invention, a biosensor with a coating formed by one or more of the processes discussed above may provide one or more layers/surfaces that provide antimicrobial properties and may additionally serve as electrodes.

In embodiments of the present invention, various materials that have antimicrobial characteristics may be utilized, including antimicrobial metals such as silver, gold, platinum, palladium, iridium, zinc, copper, tin, antimony, bismuth, or mixtures of one or more of these metals with or without other metals, and other antimicrobial agents such as rifampin, minocycline, chlorhexidine, cefazolin, teicoplanin, vancomycin, other antibiotics from the beta-lactam family, antibiotics from the quinolone family, antibiotics from the aminoglycoside family, antibiotics from other families and other antiseptic agents. Antiseptic agents are compounds that prevent the growth of microbes but do not kill them. Suitable antibiotics may include bactericidal antibiotics or bacteriostatic antibiotics. Combinations of two or more antimicrobial agents in various configurations on a biosensor may also be used successfully. Persons skilled in the art will recognize that there are also other agents that may be used to minimize microbial colonization and infection.

In an embodiment of the present invention, a sensor may be in electrical contact with a circuit board or integrated circuit in a sensor module or on-skin unit. This module may be placed on the skin of a user and may contain electrical circuitry that may amplify the sensor signal and/or may contain a battery to maintain the polarizing potential and/or may provide power to transmit the signals to a distant receiver. In an embodiment, an antimicrobial agent may be placed on the portion of a sensor module that contacts the skin near or at the site of the puncture wound created by the sensor. In another embodiment, an adhesive bandage/patch may contact the skin and the sensor module and/or the patch may contain at least one antimicrobial agent. In embodiments, an antimicrobial patch may be used separate from or in conjunction with an antimicrobial sleeve/agent on the body of the sensor. In addition or separately, a portion of an on-skin unit of a device in accordance with an embodiment of the present invention may be coated or treated with one or more antimicrobial agents as provided herein.

FIG. 2 illustrates an on-skin unit 202 for a biosensor in accordance with an embodiment of the present invention. On-skin unit 202 may include a variety of electrical network components and may further be configured to provide data to another electrically coupled unit (wired or wirelessly), such as an electronic monitoring unit. On-skin unit 202 may be attached to skin using a variety of mechanisms including adhesive or a patch 204, secured, for example, using adhesive and/or staples. In an embodiment in which patch 204 is utilized, patch 204 may be secured to on-skin unit 202 using adhesive or a suitable mechanical means (clips, snaps, rails, etc.) and patch 204 may be further secured to skin using adhesive, staples, etc. In an embodiment, a passage or port 206 through patch 204 may be provided through which a biosensor (not shown) may pass to couple with various electrical network components.

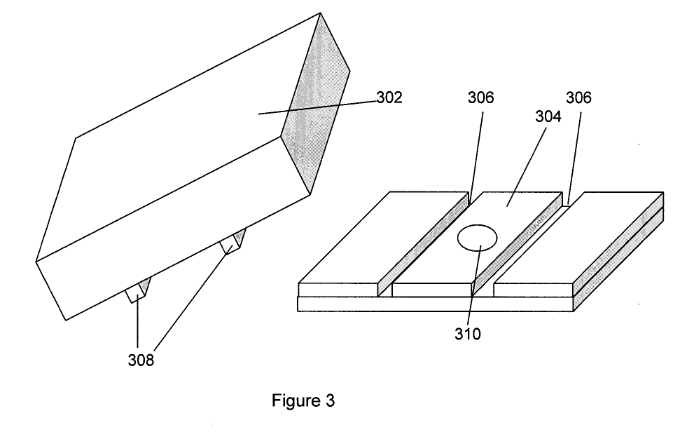

FIG. 3 illustrates an exemplary attachment mechanism to attach an on-skin unit 302 to a patch 304. Patch 304 may further utilize various attachment mechanisms for securing to skin, such as adhesive, staples, etc. Patch 304 is shown with channels 306 that correspond to rails 308 present on on-skin unit 302. In an embodiment, channels may be present on an on-skin unit and rails may be present on a patch. Channels 306 and rails 308 fit together to secure on-skin unit 302 to patch 304. In embodiments, patch 304 may be multilayered or single layered as desired. The embodiment of FIG. 3 is shown for exemplary purposes and it should be understood that other configurations of rails (number, shape, size) as well as other attachment mechanisms (such as clips, snaps, etc.) may be utilized. In an embodiment, a passage or port 310 through patch 304 may be provided through which a biosensor (not shown) may pass to enter the skin and to couple with various electrical network components in on-skin unit 302.

In an embodiment, a patch may be secured to skin by stapling the patch to the skin with bio-resorbable staples. In an embodiment, by the time the patch is ready to be removed, for example after 3-7 days (or longer), the staples may be absorbed by the body. In embodiments, suitable staples include those used to close wounds. In embodiments, staples may be configured with a two, three (or more) pronged staple geometry, or as a single fish-hook or anchor-like device that may not be removed easily, but may be absorbed by an interaction with the body in a desired period of days.

In an embodiment, an antimicrobial agent may be provided to an on-skin unit and/or a patch prior to attachment to skin. Such a patch may be surface treated with an antimicrobial agent and/or may be impregnated with an antimicrobial agent. In an embodiment using staples to secure a patch to skin, an antimicrobial agent may be applied to the staples prior to or after attachment such that the antimicrobial agent covers at least a portion of the length of each staple and/or its tines to reduce or eliminate potential for infection.

Thus, in an embodiment of the present invention there is provided a biosensor comprising an antimicrobial agent disposed on an exterior surface of at least a portion of a sensor and/or an on-skin unit and/or an associated patch. In an embodiment, an antimicrobial agent may be configured on a sensor and/or an on-skin unit and/or a patch to be in direct skin or tissue contact while in use, whether briefly or for an extended period of time.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. A biosensor, comprising:
an elongated indicating electrode circumferentially surrounded along at least a portion thereof by an insulating layer; and
a silver/silver chloride reference electrode circumferentially surrounding at least a portion of said insulating layer, wherein said silver/silver chloride reference electrode is adapted to, when in use, extend from a location above a skin surface to a location beneath the skin surface.

2. The biosensor of claim 1, wherein said elongated indicating electrode is a flexible wire-type electrode.

3. The biosensor of claim 1, wherein said elongated indicating electrode is a hollow tube constructed of metal, polymer, or glass.

4. The biosensor of claim 1, wherein said silver/silver chloride reference electrode is further coated at least partially by a further antimicrobial agent.

5. The biosensor of claim 4, wherein said antimicrobial agent circumferentially surrounds at least a portion of said silver/silver chloride reference electrode.

6. A biosensor, comprising:
an elongated indicating electrode circumferentially surrounded along at least a portion thereof by an insulating layer; and
an antimicrobial reference electrode circumferentially surrounding at least a portion of said insulating layer, wherein said antimicrobial reference electrode is adapted to, when in use, extend from a location above a skin surface to a location beneath the skin surface.

7. The biosensor of claim 6, wherein an antimicrobial property of said antimicrobial reference electrode is imparted by selecting a material for said antimicrobial reference electrode from silver, gold, platinum, palladium, iridium, zinc, copper, tin, antimony, bismuth, or mixtures thereof.

8. The biosensor of claim 6, wherein an antimicrobial property of said antimicrobial reference electrode is imparted by coating a reference electrode at least partially with an antimicrobial agent to form said antimicrobial reference electrode.

9. The biosensor of claim 8, wherein said antimicrobial agent circumferentially surrounds at least a portion of said reference electrode.

10. The biosensor of claim 6, wherein said elongated indicating electrode is a flexible wire-type electrode.

11. The biosensor of claim 6, wherein said elongated indicating electrode is a hollow tube constructed of metal, polymer, or glass.

12. An analyte sensing system, comprising:
a biosensor having an elongated indicating electrode circumferentially surrounded along at least a portion thereof by an insulating layer, and an antimicrobial reference electrode circumferentially surrounding at least a portion of said insulating layer, wherein said antimicrobial reference electrode is adapted to, when in use, extend from a location above a skin surface to a location beneath the skin surface;
an on-skin unit configured to electrically couple to said biosensor to receive a signal from said biosensor when in use.

13. The analyte sensing system of claim 12, further comprising a patch wherein said on-skin unit is further configured to couple to said patch and said patch is configured to be secured to skin when in use.

14. The analyte sensing system of claim 13, wherein said patch further comprises adhesive on one or both primary surfaces to secure said patch to said on skin-unit and/or to skin when in use.

15. The analyte sensing system of claim 13, wherein said patch and said on-skin unit comprise corresponding mechanical securing means configured to secure said on-skin unit to said patch.

16. The analyte sensing system of claim 13, wherein said patch is coated and/or impregnated with an antimicrobial agent.

17. The analyte sensing system of claim 13, further comprising bioresorbable staples configured to secure said patch to skin when in use.

18. The analyte sensing system of claim 17, wherein said bioresorbable staples are coated with an antimicrobial agent.

19. The analyte sensing system of claim 12, wherein said on-skin unit is coated on at least a portion thereof with an antimicrobial agent.

20. The analyte sensing system of claim 12, wherein an antimicrobial property of said antimicrobial reference electrode is imparted by selecting a material for said antimicrobial reference electrode from silver, gold, platinum, palladium, iridium, zinc, copper, tin, antimony, bismuth, or mixtures thereof.

21. The analyte sensing system of claim 12, wherein an antimicrobial property of said antimicrobial reference electrode is imparted by coating a reference electrode at least partially with an antimicrobial agent to form said antimicrobial reference electrode.

22. The analyte sensing system of claim 21, wherein said antimicrobial agent circumferentially surrounds at least a portion of said reference electrode.
